(19) United States
(12) Patent Application Publication
(10) Pub. No.: US 2018/0284298 A1
(43) Pub. Date: Oct. 4, 2018

(54) RADIATION CONVERSION PANEL AND TALBOT IMAGING DEVICE

(71) Applicant: KONICA MINOLTA, INC., Tokyo (JP)

(72) Inventors: Tadashi ARIMOTO, Tokyo (JP); Mitsuko MIYAZAKI, Tokyo (JP)

(73) Assignee: KONICA MINOLTA, INC., Tokyo (JP)

(21) Appl. No.: 15/840,609

(22) Filed: Dec. 13, 2017

(30) Foreign Application Priority Data
Mar. 28, 2017 (JP) 2017-063071

(51) Int. Cl. G01T 1/20

(52) U.S. Cl. CPC G01T 1/2006; A61B 6/48; G01T 1/2002

(57) ABSTRACT

A radiation conversion panel includes: a scintillator panel having a sectioned structure; and a photoelectric conversion panel, the scintillator panel and the photoelectric conversion panel being disposed so as to be opposed to each other, the scintillator having a width smaller than a width of the non-light receiver present in the photoelectric conversion panel, wherein a layer made of a light transmissive material is disposed between the scintillator panel and the photoelectric conversion panel.

The entire disclosure of Japanese patent Application No. 2017-063071, filed on Mar. 28, 2017, is incorporated herein by reference in its entirety.

### BACKGROUND

### Technological Field

The present invention relates to a radiation conversion panel and a Talbot imaging device expected to be used in a next-generation Talbot system.

### Description of the Related art

Currently, in X-ray image diagnosis, an absorptive image that images the attenuation of an X-ray after passing through an object is used. On the other hand, since an X ray is a type of electromagnetic waves, attention has been paid to this wave nature and attempts have been recently made to image changes in the phase after passing through an X-ray object. These are called absorption contrast and phase contrast, respectively. An imaging technique using this phase contrast has higher sensitivity to a light element than the conventional absorption contrast; therefore, it is thought that the sensitivity to the soft tissue of the human body which contains many light elements is high.

However, the conventional phase contrast imaging technique requires the use of a synchrotron X-ray source and a minute focus X-ray tube; therefore, it has been thought that practical use in general medical facilities is difficult because the former requires a huge facility and the latter cannot secure sufficient X-ray dose to photograph the human body.

In order to solve this problem, X-ray image diagnosis (Talbot system) using an X-ray Talbot-Lau interferometer, which can acquire a phase contrast image by using an X-ray source used in a medical field in the past, is expected.

In a Talbot-Lau interferometer, as shown in FIG. 2, a G0 lattice, a G1 lattice, and a G2 lattice are disposed between a medical X-ray tube and an FPD, respectively, and the refraction of the X-ray by the subject is visualized as moire fringes. An X-ray is applied in a longitudinal direction from an X-ray source arranged in an upper part and reaches an image detector through G0, a subject, G1, and G2.

As a manufacturing method of the lattice, example, a method is known in which a silicon wafer having high X-ray transparency is etched to provide a lattice-shaped recessed portion and a heavy metal having a high X-ray shielding property is filled in the recessed portion.

However, in the above method, it is difficult to increase the area due to the size of available silicon wafer, constraints of etching equipment, and the like, and an object to be photographed is limited to a small part. Furthermore, it is not easy to form a deep recess in a silicon wafer by etching, and it is also difficult to evenly fill the metal to the depth of the recess, so that it is difficult to fabricate a lattice having a thickness enough to shield an X-ray sufficiently. For this reason, under a high-voltage shooting condition, an X-ray penetrates the lattice, making it impossible to obtain a good image.

Therefore, as shown in FIG. 3, it is also considered to adopt a scintillator having a sectioned structure (a slit-shaped scintillator in FIG. 3) in the scintillator constituting the image detector by removing the G2 lattice.

As a sectioned scintillator, for example, JP 5127246 B2 discloses a detection element manufactured by filling a polymer containing nanoparticles made of a scintillation material in a lattice cavity (groove) manufactured by an etching technique.

Furthermore, “Applied Physics Letter 98, 171107 (2011)” Structured scintillator for x-ray grating interferometry “(Paul Scherrer Institute (PSI))” discloses a lattice-shaped scintillator in which a groove of a lattice fabricated by etching a silicon wafer is filled with a phosphor (CsI) is disclosed.

By causing a sectioned scintillator to face a photoelectric conversion panel, the emission of the scintillator by radiation is converted into an electric signal to obtain a digital image. The sensor pixels constituting the photoelectric conversion panel include, in addition to a light receiver that senses the light emission of the scintillator, a non-light receiver made up of a TFT element, wiring and the like. In order to improve the resolution, as the width of the sectioned scintillator is miniaturized, as shown in FIG. 4, a portion where light emission of the scintillator is not received by the sensor is formed. In this case, there is a problem that the light emission of the scintillator is not transmitted to the sensor and the image quality is deteriorated. Even when a plurality of photoelectric conversion panels is tiled and used, the joints of the panels serve as non-light receivers, and the same problem occurs.

### SUMMARY

Under such circumstances, the inventors of the present invention have conducted intensive studies and, as a result, by providing a layer made of a light transmissive material between the sectioned scintillator and a photoelectric conversion element, the light emission of the scintillator is easily transmitted to the sensor, and the image quality is improved, thus completing the present invention. An object of the present invention is to provide a radiation conversion panel and a Talbot imaging device.

To achieve the abovementioned object, according to an aspect of the present invention, a radiation conversion panel reflecting one aspect of the present invention comprises: a scintillator panel having a sectioned structure; and a photoelectric conversion panel, the scintillator panel and the photoelectric conversion panel being disposed so as to be opposed to each other, the scintillator having a width smaller than a width of a non-light receiver present in the photoelectric conversion panel, wherein a layer made of a light transmissive material is disposed between the scintillator panel and the photoelectric conversion panel.

### BRIEF DESCRIPTION OF THE DRAWINGS

The advantages and features provided by one or more embodiments of the invention will become more fully understood from the detailed description given hereinbelow and the appended drawings which are given by way of illustration only; and thus are not intended as a definition of the limits of the present invention:

FIG. 1 is a schematic view showing an example of a radiation conversion panel according to an embodiment of the present invention;

FIG. 2 is a schematic configuration view of an example of a Talbot imaging device;

FIG. 3 is a schematic configuration view of an example of a Talbot imaging device;

FIG. 4 is a schematic explanatory view of a non-light receiver in a photoelectric conversion element.

FIG. 5 is a schematic view of a slit-shaped scintillator; and

FIG. 6 is a schematic view of an example of a slit-shaped scintillator.

### DETAILED DESCRIPTION OF EMBODIMENTS

Hereinafter, one or more embodiments of the present invention will be described with reference to the drawings. However, the scope of the invention is not limited to the disclosed embodiments.

A radiation conversion panel of an embodiment of the present invention will be described.

The radiation conversion panel includes: a scintillator panel having a sectioned structure; and a photoelectric conversion panel, the scintillator panel and the photoelectric conversion panel being disposed so as to be opposed to each other, the scintillator having a width smaller than a width of a non-light receiver present in the photoelectric conversion panel, wherein a layer made of a light transmissive material is disposed between the scintillator panel and the photoelectric conversion panel.

Scintillator Panel Having Sectioned Structure

The scintillator panel having the sectioned structure includes a flat plate-like substrate having radiation transparency, a partition wall structure portion having a section of a lattice-shaped unit provided on the substrate, and a scintillator layer filled with a phosphor in each of the sections.

The width of the scintillator in an embodiment of the present invention means the shortest length in a direction perpendicular to radiation in the sectioned scintillator. When the sectioned scintillator is a cylinder, the width of the scintillator corresponds to the diameter, and in the case of a rectangular parallelepiped, the width of the scintillator corresponds to the length of the side at the bottom. In addition, when the sectioned scintillator has a slit shape, the width of the scintillator corresponds to the thickness of the scintillator layer. In the case where the scintillator has an inclined structure, the average value on the incident side and the outgoing side is taken as the width of the scintillator. The width of the scintillator is preferably 0.1 to 100 μm, more preferably 0.5 to 50 μm, and still more preferably 1.0 to 10 μm.

A plurality of scintillator panels having a sectioned structure may be tiled.

A substrate having radiation transparency is a plate-like body capable of supporting the scintillator, and various kinds of glasses, polymer materials, metals, and the like can be used.

For example, glass sheets such as quartz, borosilicate glass, and chemically strengthened glass, ceramic substrates such as sapphire, silicon nitride and silicon carbide, a semiconductor substrate such as silicon, germanium, gallium arsenide, gallium phosphorus, and gallium nitrogen, or polymer films (plastic film) such as cellulose acetate film, polyester film, polyethylene terephthalate film, polyamide film, polyimide film, triacetate film, polycarbonate film, and carbon fiber reinforced resin sheet, metal sheets such as an aluminum sheet, an iron sheet, a copper sheet or the like, or a metal sheet having a coating layer of the metal oxide can be used. A material having a high elastic modulus and a stable thermal expansion coefficient like a glass sheet material is preferable.

Specific examples of the polymer film include a polymer film including polyethylene naphthalate, polyethylene terephthalate, polybutylene naphthalate, polycarbonate, syndiotactic polystyrene, polyether imide, polyarylate, polysulfone, polyethersulfone or the like. These may be used singly or in lamination or mixing. Among them, as a particularly preferable polymer film, a polymer film containing polyimide or polyethylene naphthalate is preferable as described above.

Such a scintillator panel can be produced with reference to JP 2011-21924 A.

That is, a glass paste which is a mixture of a pigment or ceramic powder and a low melting point glass powder is coated at a predetermined thickness by screen printing on a flat plate shaped substrate having radiation transparency, and the coated glass paste is dried to form a bottom of a barrier rib structure (first step). Thereafter, the glass paste is applied by screen printing using a lattice pattern having a size determined by the number of pixels in a lattice shape with a predetermined pitch, an opening with a predetermined size, and a predetermined thickness in pixel units in vertical and horizontal directions, and subsequently, drying is also carried out. This is repeated a plurality of times to form a partition wall of a predetermined height. Thereafter, firing is performed in the air at 550° C. to form a partition wall structure portion having each section of a space partitioned by the bottom portion and the partition wall on the substrate (third step). Then, the partition wall structure portion is filled with a phosphor to form a scintillator layer, and a scintillator panel having a sectioned structure is manufactured (fourth step).

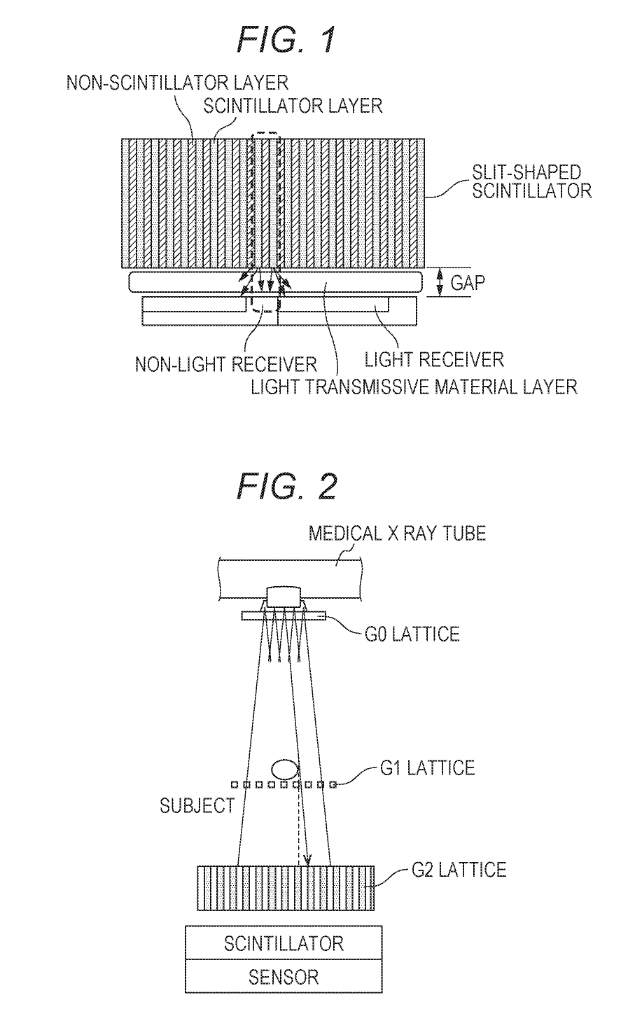

In an embodiment of the present invention, as a preferred embodiment of the scintillator having a sectioned structure, as shown in FIG. 1, a slit-shaped scintillator having a structure in which the scintillator layer and the non-scintillator layer are repeatedly laminated in a direction substantially parallel to a radiation incidence direction can be cited. Substantially parallel is almost parallel, and even if it is perfectly parallel and there are some inclination and curvature, it is included in the almost parallel category. Such a slit-shaped scintillator can also have a large area. As shown in FIG. 1, in the case of the slit-shaped scintillator, the thickness of the scintillator layer in a lamination direction corresponds to the width of the scintillator.

The width of the scintillator is appropriately selected according to the purpose and the configuration of the sectioned scintillator, and is approximately 0.25 to 200 μm, but not limited thereto.

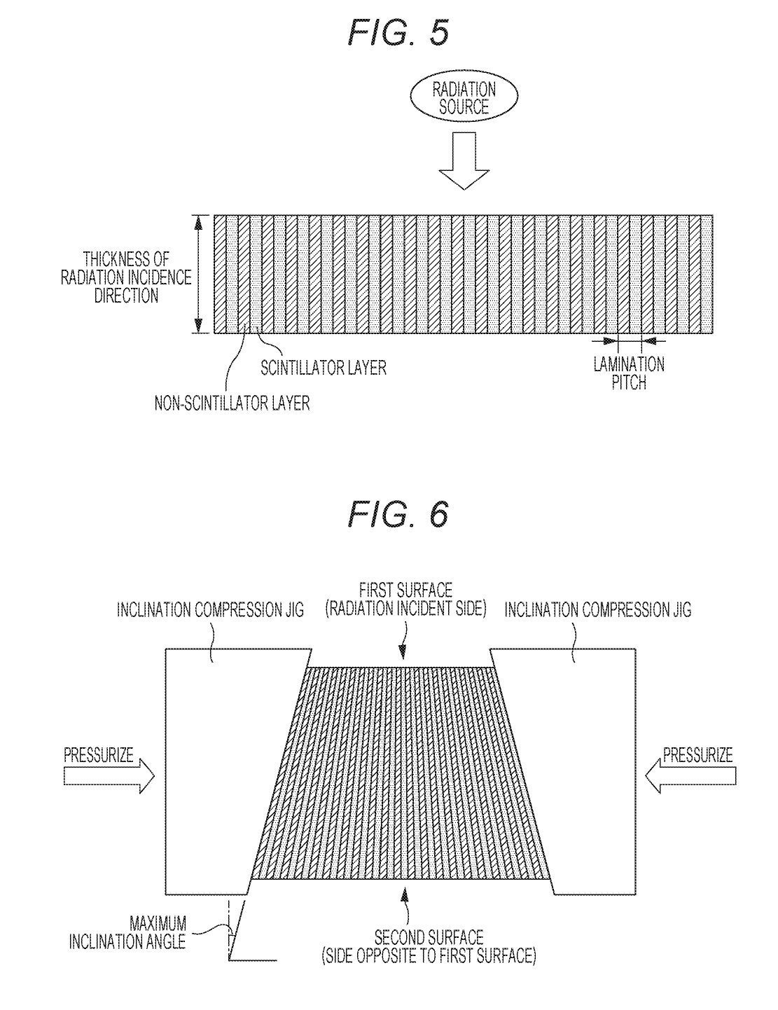

FIG. 5 shows an enlarged view of the slit-shaped scintillator. As shown in FIG. 5, a ratio (duty ratio) of the thickness (hereinafter referred to as lamination pitch) of the pair of scintillator layers and the non-scintillator layer in the lamination direction to the thickness of the scintillator layer and the thickness of the non-scintillator layer in the lamination direction (hereinafter duty ratio) are derived from the Talbot interference condition. The lamination pitch is preferably from 0.2 to 200 μm, more preferably from 1.0 to 100 μm, and still more preferably from 2.0 to 20 μm. The duty ratio is preferably from 30/70 to 70/30. In order to obtain a diagnostic image of a sufficient area, it is preferable that the number of repeated lamination layers of the laminated pitch is 1,000 to 500,000.

The thickness of a slit scintillator panel in a radiation incidence direction in an embodiment of the present invention is preferably 10 to 1,000 μm, and more preferably 100 to 500 μm. When the thickness in the radiation incidence direction is smaller than the lower limit value of the above range, the light emission intensity of the scintillator is weakened and image quality is deteriorated. In addition, when the thickness in the radiation incidence direction is greater than the upper limit of the range, the distance which the light emitted from the scintillator reaches the photoelectric conversion panel increases, so that light easily diffuses and sharpness deteriorates.

The scintillator layer includes a layer containing a scintillator as a main component, and preferably contains scintillator particles. As the scintillator according to an embodiment of the present invention, a substance capable of converting radiation such as an X ray into a different wavelength such as visible light can be appropriately used. In particular, scintillators and phosphors described in “Phosphor Handbook” (edited by the Society of Phosphors, Ohmsha, Ltd., 1987) from pages 284 to 299, and substances described in the web site “Scintillation Properties (http://scintillator.lbl.gov/)” of Lawrence Berkeley National Laboratory in the United States may be considered. However, even a substance not pointed out here can be used as a scintillator as long as the substance is “a substance capable of converting radiation such as an X ray into a different wavelength such as visible light”.

Specific examples of the composition of the scintillator include the following examples. First, metal halide phosphors represented by the basic composition formula (I): M_{I}X.aM_{II}X′₂.bM_{III}X″₃:zA can be included.

In the basic composition formula (I), M_{I} represents at least one selected from the group consisting of elements that can be a monovalent cation, that is, lithium (Li), sodium(Na), potassium (K), rubidium (Rb), cesium (Cs), thallium (Tl), and silver (Ag).

M_{II} represents at least one selected from the group consisting of elements that can be divalent cations, that is, beryllium (Be), magnesium (Mg), calcium (Ca), strontium (Sr), barium (Ba), nickel (Ni), copper (Cu), zinc (Zn), and cadmium (Cd).

M_{III} represents at least one selected from the group consisting of elements belonging to scandium (Sc), yttrium (Y), aluminum (Al), (Ga), indium (In), and lanthanoid.

X, X′ and X″ each represent a halogen element; however, each of them may be different element or the same element.

A represents at least one element selected from the group consisting of Y, Ce, Pr, Nd, Sm, Eu, Gd, Tb, Dy, Ho, Er, Tm, Yb, Lu, Na, Mg, Cu, Ag (silver), Tl and Bi (bismuth).

a, b and z each independently represent a numerical value within the range of 0≤a≤0.5, 0≤b<0.5, and 0<z<1.0.

Furthermore, rare earth activated metal fluorohalide phosphor represented by basic composition formula (II): M_{II}FX:zLn can also be included.

In the basic composition formula (II), M_{II} represents at least one alkaline earth metal element, Ln represents at least one element belonging to lanthanoid, and X represents at least one halogen element. Furthermore, z is 0<z≤0.2.

In addition, rare earth oxysulfide phosphors represented by basic composition formula (III): Ln₂O₂S:zA can also be included.

In the basic composition formula Ln represents at least one element belonging to lanthanoid, and A represents at least one element selected from the group consisting of Y, Ce, Pr, Nd, Sm, Eu, Gd, Tb, Dy, Ho, Er, Tm, Yb, Lu, Na, Mg, Cu, Ag (silver), Tl and Bi (bismuth). Furthermore, z is 0<z<1.

By using terbium (Tb), dysprosium (Dy) or the like as the element type of A, in particular, Gd₂O₂S using gadolinium (Gd) as Ln is preferable because it is known that the sensor panel shows high luminescence property in the wavelength range Where light is most likely to be received.

In addition, a metal sulfide-based phosphor represented by the basic composition formula (IV): M_{II}S:zA can also be included.

In the basic composition formula (IV), M_{II} represents at least one element selected from the group consisting of elements that can be divalent cations, that is, alkaline earth metals, Zn (zinc), Sr (strontium), Ga (gallium), and the like, and A represents at least one element selected from the group consisting of Y, Ce, Pr, Nd, Sm, Eu, Gd, Tb, Dy, Ho, Er, Tm, Yb, Lu, Na, Mg, Cu, Ag (silver), Tl and Bi (bismuth). Furthermore, z is 0<z<1.

In addition, a metal oxoacid salt-based phosphor represented by the basic composition formula (V): M_{IIa}(AG)_{b}zA can also be included.

In the basic composition formula (V), M_{II} represents a metal element that can be a cation, (AG) represents at least one oxo acid group selected from the group consisting of phosphate, borate, silicate, sulfate, tungstate and aluminate, and A represents at least one element selected from the group consisting of Y, Ce, Pr, Nd, Sm, Eu, Gd, Tb, Dy, Ho, Er, Tm, Yb, Lu, Na, Mg, Cu, Ag (silver), Tl and Bi (bismuth).

“a” and “b” represent all possible values depending on the valence of the metal and oxo acid groups. z is 0<z<1.

Further, a metal oxide-based phosphor represented by the basic composition formula (VI): MₐO_{b}:zA can also be included.

In the basic composition formula (VI), M represents at least one element selected from metallic elements that can be a cation, particularly a metal belonging to lanthanoid is preferable. Specific examples include GD₂O₃ and Lu₂O₃.

A represents at least one element selected from the group consisting of Y, Ce, Pr, Nd, Sm, Eu, Gd, Tb, Dy, Ho, Er, Tm, Yb, Lu, Na, Mg, Cu, Ag (silver), Tl and Bi (bismuth).

“a” and “b” represent all possible values depending on the valence of the metal and oxo acid groups. z is 0<z<1.

Besides, a metal acid halide-based phosphor represented by the basic composition formula (VII): LnOX:zA can also be included.

In the basic composition formula (VII), Ln represents at least one element belonging to lanthanoid, X represents at least one halogen element, and A represents at least one element selected from the group consisting of Y, Ce, Pr, Nd, Sm, Eu, Gd, Tb, Dy, Ho, Er, Tm, Yb, Lu, Na, Mg, Cu, Ag (silver), Tl and Bi (bismuth). Also, z is 0<z<1.

It is preferable that the scintillator particles each contain at least either CsI or GD₂O₂S as a main component.

The average particle diameter of the scintillator particles is selected according to the thickness of the scintillator layer in the lamination direction and is preferably 100% or less, and more preferably 90% or less, with respect to the thickness in the lamination direction of the scintillator layer. When the average particle size of the scintillator particles exceeds the above range, the disorder of the laminated pitch becomes large and the Talbot interference function decreases.

The content percentage of the scintillator particles in the scintillator layer is preferably 30 vol % or more, more preferably 50 vol % or more, and still more preferably 70 vol % or more in consideration of luminescence property.

Two or more scintillator particles may be contained in the scintillator layer, or two or more scintillator layers containing different scintillator particles may be combined.

The non-scintillator layer in an embodiment of the present invention is a layer that transmits visible light and does not contain a scintillator as a main component, and the content of the scintillator in the non-scintillator layer is less than 10 vol %, preferably less than 1 vol %, most preferably 0 vol %.

Above all, a material transparent to the emission wavelength of the scintillator is particularly preferable.

By making the non-scintillator layer transparent, the light emitted from the scintillator propagates not only within the scintillator layer but also into the non-scintillator layer, thereby increasing the amount of light reaching the sensor and improving brightness. The transmittance in the laminating direction of the non-scintillator layer single layer is 80% or more, preferably 90%, and more preferably 95% or more.

It is desirable for the non-scintillator layer to contain, as a main component, various glasses, polymer materials, and the like having the above described transmittance. These may be used singly or may be used in combination of a plurality of them.

Glass sheets such as quartz, borosilicate glass, chemically strengthened glass and the like; ceramics such as sapphire; polyesters such as polyethylene terephthalate (PET) and polyethylene naphthalate (PEN), aliphatic polyamides such as nylon, aromatic polyamide (aramid), polyimide, polyamideimide, polyetherimide, polyethylene, polypropylene, polycarbonate, celluloses such as cellulose diacetate (DAC), cellulose triacetate (TAC), cellulose acetate butyrate (CAB), and cellulose acetate propionate (CAP) epoxy, bismaleimide, polylactic acid, sulfur-containing polymers such as polyphenylene sulfide and polyether sulfone, polymers such as polyether ether ketone, fluororesin, acrylic resin, and polyurethane; bio-nanofibers containing chitosan, cellulose and the like such as glass fiber (in particular, fiber reinforced resin sheet containing these fibers) can be used.

As a non-scintillator layer, a polymer film is preferable from the viewpoint of handling. A commercially available polymer film may be used, or a polymer film may be formed on a separator film having releasability and then peeled off from the separator film and used. Fine particles of silica or the like may be contained in the polymer film for the purpose of preventing blocking and improving slipperiness during transportation.

In an embodiment of the present invention, it is possible to perform lamination by joining the scintillator layer and the non-scintillator layer. Joining in an embodiment of the present invention means bonding the scintillator layer and the non-scintillator layer to integrate them. An adhesive layer may be interposed between the scintillator layer and the non-scintillator layer, and the adhesive resin may be previously contained in the scintillator layer or the non-scintillator layer so that the scintillator layer and the non-scintillator layer may be joined without interposing the adhesive layer.

The adhesive resin may be contained in any of the scintillator layer and the non-scintillator layer, but it is particularly preferable that the scintillator layer contains an adhesive resin as a binder of the scintillator particles. In addition, it is preferable that the adhesive resin is a material transparent to the emission wavelength of the scintillator so as not to inhibit the propagation of light emitted from the scintillator.

The adhesive resin is not particularly limited as long as the object of an embodiment of the present invention is not impaired.

For example, the adhesive resin may include natural polymeric substances such as protein such as gelatin, polysaccharide such as dextran, or guru arabic; and synthetic polymer substances such as polyvinyl butyral, polyvinyl acetate, nitrocellulose, ethylcellulose, vinylidene chloride-vinyl chloride copolymer, poly (meth) acrylate, vinyl chloride-vinyl acetate copolymer, polyurethane, cellulose acetate butyrate, polyvinyl alcohol, polyester, epoxy resin, polyolefin resin, polyamide resin and the like. These resins may be crosslinked with crosslinking agents such as epoxy or isocyanate, and these adhesive resins may be used singly or in combination of two or more. The adhesive resin may be either a thermoplastic resin or a thermosetting resin.

When the adhesive resin is contained in the scintillator layer, the content percentage is preferably 1 to 70 vol %, more preferably 5 to 50 vol %, and further preferably 10 to 30 vol %. If the content percentage is lower than the lower limit of the above range, sufficient adhesiveness cannot be obtained, and conversely, when the content percentage is higher than the upper limit of the above range, the content percentage of the scintillator becomes insufficient and the amount of luminescence decreases.

As a method of forming the scintillator layer, a composition prepared by dissolving or dispersing the scintillator particles and the adhesive resin in a solvent may be coated, and a composition prepared by heating and melting a mixture containing the scintillator particles and the adhesive resin may be coated.

When coating a composition in which the scintillator particles and the adhesive resin are dissolved or dispersed in a solvent, examples of usable solvents include lower alcohols such as methanol, ethanol, isopropanol and n-butanol, ketones such as acetone, methyl ethyl ketone, methyl isobutyl ketone, cyclohexanone and the like, esters of lower fatty acids such as methyl acetate, ethyl acetate and n-butyl acetate with lower alcohols, ethers such as dioxane, ethylene glycol monoethyl ether, ethylene glycol monomethyl ether, aromatic compounds such as triol, xylene and the like, halogenated hydrocarbons such as methylene chloride and ethylene chloride, and mixtures thereof. In the composition, various additives such as a dispersant for improving the dispersibility of the scintillator particles in the composition, and a curing agent and a plasticizer for improving the bonding force between the adhesive resin and the scintillator particles in the scintillator layer after formation may be mixed.

Examples of the dispersant used for such purpose include phthalic acid, stearic acid, caproic acid, lipophilic surfactant, and the like.

Examples of the plasticizer may include phosphoric acid esters such as triphenyl phosphate, tricresyl phosphate, diphenyl phosphate and the like; phthalic acid esters such as diethyl phthalate and dimethoxyethyl phthalate; glycolic acid esters such as ethyl phthalyl ethyl glycolate and butyl phthalyl butyl glycolate; polyester of triethylene glycol and adipic acid, Polyester of polyethylene glycols and aliphatic dibasic acids such as polyethylene of diethylene glycol and succinic acid and the like. As the curing agent, a known curing agent for a thermosetting resin can be used.

When heating and melting the mixture containing the scintillator particles so as to be coated, it is preferable to use hot melt resin as the adhesive resin. For example, the hot melt resin formed of resins such as polyolefin-based resin, polyimide, polyester-based resin, polyurethane-based resin or acrylic resin, as a main component, can be used. Among these, from the viewpoints of light permeability, moisture resistance and adhesiveness, hot melt resin including polyolefin-based resin as a main component is preferable. As the polyolefin-based resin, for example, ethylene-vinyl acetate copolymer (EVA), ethylene-acrylic acid copolymer (EAA), ethylene-acrylate copolymer (EMA), ethylene-methacrylic acid copolymer (EMAA), ethylene-methacrylic acid ester copolymer (EMMA), an ionomer resin or the like can be used. It is to be noted that these resins may be used as a so-called polymer blend obtained by combining two or more kinds.

There are no particular restrictions on the means for coating the composition for forming the scintillator layer; however, usual coating means such as a doctor blade, a roll coater, a knife coater, an extrusion coater, a die coater, a gravure coater, a lip coater, a capillary coater, a bar coater, or the like can be used.

The slit-shaped scintillator panel is produced by repeatedly laminating a scintillator layer and a non-scintillator layer, and then joining the adjacent layers.

There is no particular restriction on the method of repeatedly laminating the scintillator layer and the non-scintillator layer; however, the individually formed scintillator layer and the non-scintillator layer may be divided into a plurality of sheets and then alternately repeatedly laminated.

In an embodiment of the present invention, since it is easy to adjust the number of laminated layers and the thickness of the laminate, it is preferable to form a plurality of partial laminates in which the scintillator layer and the non-scintillator layer are joined, and then laminate the plurality of partial laminates to form the laminate.

For example, a partial laminate including a pair of scintillator layers and a non-scintillator layer may be formed in advance, and the partial laminate may be divided into a plurality of sheets and laminated repeatedly.

If the partial laminate including the scintillator layer and the non-scintillator layer has a film shape capable of being wound up, the partial laminate can be efficiently laminated by winding the partial laminate on the core. The winding core may be cylindrical or flat. More efficiently, the repeated laminates of the scintillator layer and the non-scintillator layer produced by the above method may be joined (integrated) by pressurization, heating, or the like, and then divided into a plurality of sheets. Thereafter, the divided sheets may be repeatedly laminated.

There is no particular restriction on a method for forming the partial laminate including the scintillator layer and the non-scintillator layer; however, the scintillator layer may be formed by selecting a polymer film as the non-scintillator layer and coating a composition containing scintillator particles and an adhesive resin on one side thereof. Furthermore, a composition containing scintillator particles and adhesive resin may be coated on both sides of the polymer film.

As described above, when the partial laminate is formed by coating the composition containing the scintillator particles and the adhesive resin on the polymer film, it is possible to simplify the step and to easily divide the partial laminate into a plurality of sheets. A dividing method is not particularly limited, and an ordinary cutting method is selected.

Alternatively, a transfer substrate coated with the scintillator layer in advance may be transferred onto a film including the non-scintillator layer. The transfer substrate is desorbed by means such as peeling as necessary.

In an embodiment of the present invention, the scintillator layer and the non-scintillator layer are joined by pressurizing the laminate so that the scintillator layer and the non-scintillator layer are substantially parallel to the radiation incidence direction.

By heating the repeated laminate of a plurality of scintillator layers and non-scintillator layers in a state pressurized to a desired size, it is possible to adjust the laminated pitch to a desired value.

There is no particular restriction on a method for pressurizing the repeated laminate of the plurality of scintillator layers and the non-scintillator layers to have a desired size; however, it is preferable to apply pressure in a state where a spacer such as a metal is provided in advance so that the laminate is not compressed to a desired size or more. The pressure at that time is preferably 1 MPa to 10 GPa. If the pressure is lower than the lower limit of the above range, the resin component contained in the laminate may not be deformed to a predetermined size. When the pressure is higher than the upper limit value of the above range, the spacer may be deformed, and there is a possibility that the laminate is compressed to a desired dimension or more.

By heating the laminate in a pressurized state, joining can be made more robust.

Conditions for heating the repeated laminate of the plurality of scintillator layers and the non-scintillator layers depend on the kind of the resin; however, it is preferable to heat the repeated laminate at a temperature equal to or higher than the glass transition point for thermoplastic resin and at a temperature equal to or higher than curing temperature for thermosetting resin, for about 0.5 to 24 hours. The heating temperature is preferably 40° C. to 250° C. in general. If the temperature is lower than the lower limit of the above range, the fusion or curing reaction of the resin may be insufficient in some cases. There is a possibility of poor connection or returning to the original size when releasing compression. If the temperature is higher than the upper limit of the above range, there is a possibility that the resin deteriorates and the optical characteristics are impaired. There is no particular restriction on the method of heating the laminate under pressure; however, a press equipped with a heating element may be used, a laminate may be heated in an oven while being sealed in a box-like jig so as to have a predetermined size, or a heating element may be mounted on a box-shaped jig.

As a state before the repeated laminate of the plurality of scintillator layers and non-scintillator layers is pressurized, it is preferable that voids exist in the interior of the scintillator layer, inside the non-scintillator layer, or in the interface between the scintillator layer and the non-scintillator layer. If pressurization is carried out in the absence of any voids, a part of the constituent material may flow out from the laminated end face to cause disorder in the laminated pitch or return to the original size when releasing the pressure. If voids are present, even if pressurized, the voids become a cushion. The laminate can be adjusted to an arbitrary size as long as the voids are zero, that is, the laminated pitch can be adjusted to an arbitrary value. The porosity is calculated from the following formula using the actual measured volume (area×thickness) of the laminate, and the theoretical volume weight/density) of the laminate.

(Actual measured volume of laminate−theoretical volume of laminate)/theoretical volume of laminate×100

If the area of the laminate is constant, the porosity is calculated from the following formula using the actual measured thickness of the laminate and the theoretical thickness (weight/density/area) of the laminate.

(Actual measured thickness of laminate−theoretical thickness of laminate)/theoretical thickness of laminate×100

The porosity of the scintillator layer after heating is preferably 30 vol % or less. When the porosity exceeds the above range, the packing ratio of the scintillator decreases and the luminance decreases.

As means for providing voids in the scintillator layer or the non-scintillator layer, for example, bubbles may be contained in the layer in the process of manufacturing the scintillator layer or the non-scintillator layer, or hollow polymer particles may be added in the layer. On the other hand, even when irregularities exist on the surface of the scintillator layer or the non-scintillator layer, since voids are formed at a contact interface between the scintillator layer and the non-scintillator layer, the same effect can be obtained. As means for providing irregularities on the surfaces of the scintillator layer and the non-scintillator layer, for example, irregularity treatment such as blast treatment or embossing treatment may be applied to the surface of the layer, and irregularities may be formed on the surface by containing a filler in the layer. When the scintillator layer is formed by coating a composition containing scintillator particles and the adhesive resin on the polymer film, irregularities are formed on the surface of the scintillator layer, and voids can be formed at the contact interface with the polymer film. The size of the irregularity can be arbitrarily adjusted by controlling the particle size and dispersibility of the filler.

Since a radiation source emitting radiation such as an X-ray is generally a point wave source, when individual scintillator layers and non-scintillator layers are formed completely in parallel, an X-ray obliquely enters in the peripheral region of a laminated scintillator. As a result, in the peripheral region, so-called eclipse occurs in which radiation does not sufficiently penetrate. The eclipse becomes a serious problem as the scintillator becomes larger in area.

In a laminated scintillator panel, when a radiation incident side is a first surface and a side opposite to the first surface is a second surface, by setting the laminated pitch of the scintillator layer and the non-scintillator layer on the second surface to be larger than the lamination pitch of the scintillator layer and the non-scintillator layer on the first surface, the individual scintillator layers and non-scintillator layers are placed so that the individual scintillator layers and non-scintillator layers are parallel to the radiation; therefore, the present problem can be improved. Specifically, by curving the laminated scintillator panel, or by causing the laminated scintillator panel to have an inclined structure without curving, the present problem can be improved. In an embodiment of the present invention, by causing the first surface and the second surface of the inclined laminated scintillator panel to be flat, the first surface and the second surface can be reasonably brought into close contact with a photoelectric conversion panel that is generally rigid and flat, which is preferable from the viewpoint of image quality improvement. On the other hand, in the case of curving the laminated scintillator panel, the laminated scintillator panel is preferable to be a flexible material since the photoelectric conversion panel also needs to follow up.

In order to cause the laminated scintillator panel to have an inclined structure, for example, in the step of pressurizing the repeated laminate of the plurality of scintillator layers and the non-scintillator layers, by making the pressing direction oblique, an inclined structure having a trapezoidal cross section can be formed. An inclination angle is maximized at the edge side of the laminated scintillator panel and continuously becomes parallel to the center. The maximum inclination angle is determined by the size of the laminated scintillator panel and the distance between the laminated scintillator panel and a radiation source, but is generally 0 to 10°. As a pressing method for forming the inclined structure, for example, a pressing jig having a predetermined inclination as shown in FIG. 6 may be used. Note that the inclination angle 0° is parallel and the above range is included in the concept of “substantially parallel” in the specification of the present application.

In an embodiment of the present invention, it is preferable to flatten a joining end face where the plurality of scintillator layers and the non-scintillator layers are joined. In particular, scattering of the scintillator light at the joining end face can be suppressed by flattening the face on the radiation incident side, the opposite side, or both sides, thereby improving the sharpness. There is no particular limitation on a flattening method, and energy such as ions, plasma, electron beam, and the like may be applied in addition to machining such as cutting, grinding, and polishing. In the case of machining, it is preferable to perform machining process in a direction parallel to the laminated structure so as not to damage the laminated structure of the scintillator layer and the non-scintillator layer.

Since the thickness of the laminated scintillator panel in an embodiment of the present invention in the radiation incidence direction is as very thin as several millimeters or less, in order to maintain the laminated structure, it is preferable that the surface on the radiation incident side, the side opposite thereto, or both surfaces are bonded and held on a support.

As the support, various glasses, polymer materials, metals and the like which can transmit radiation such as an X ray can be used; however, for example, glass sheets such as quartz, borosilicate glass, and chemically strengthened glass, ceramic substrates such as sapphire, silicon nitride and silicon carbide, a semiconductor substrate (photoelectric conversion panel) such as silicon, germanium, gallium arsenide, gallium phosphorus, and gallium nitrogen, or polymer films (plastic film) such as cellulose acetate film, polyester film, polyethylene terephthalate film, polyamide film, polyimide film, triacetate film, and polycarbonate film, metal sheets such as an aluminum sheet, an iron sheet, a copper sheet or the like, or a metal sheet having a coating layer of the metal oxide, a carbon fiber reinforced resin (CFRP) sheet, an amorphous carbon sheet or the like can be used. The thickness of the support is preferably 50 μm to 2,000 μm, and more preferably 50 to 1,000 μm.

Photoelectric Conversion Panel

The photoelectric conversion panel included in a radiation detector according to an embodiment of the present invention has a function of absorbing emitted light generated in the scintillator layer, converting the absorbed emitted light into a form of electric charge to convert it into an electric signal, and outputting information included in the emitted light as an electric signal to the outside of the radiation detector. The photoelectric conversion panel is not particularly limited as long as the photoelectric conversion panel can perform such a function, and the photoelectric conversion panel can be conventionally known.

In the photoelectric conversion panel, a photoelectric conversion element is incorporated in a panel. The configuration of the photoelectric conversion panel is not particularly limited, but normally, a photoelectric conversion panel substrate, an image signal output layer, and the photoelectric conversion element are stacked in this order.

The photoelectric conversion element may have any specific structure as long as the photoelectric conversion element has a function of absorbing light generated in the scintillator layer and converting the absorbed light into a form of electric charge. For example, the photoelectric conversion element may include a transparent electrode, a charge generation layer excited by incident light to generate electric charge, and a counter electrode. Any of these transparent electrodes, charge generation layer and counter electrode can be conventionally known. In addition, the photoelectric conversion element may include a suitable photosensor, and for example, may be obtained by two-dimensionally arranging a plurality of photodiodes. Alternatively, the photoelectric conversion element may be a two-dimensional photosensor such as a charge coupled device (CCD) or a complementary metal-oxide-semiconductor (CMOS) sensor.

The image signal output layer has a function of accumulating the electric charge obtained by the photoelectric conversion element and outputting a signal based on the accumulated electric charge. The image signal output layer may have any structure as long as the image signal output layer has the above-described function, and for example, may include a capacitor which is a charge storage element that accumulates a charge generated by the photoelectric conversion element for each pixel, and a transistor which is an image signal output element that outputs the accumulated charge as a signal. Here, a thin film transistor (TFT) is an example of a preferable transistor.

It is also possible to use a photo-counting type radiation image detector as the above-described radiation image detector. The photo-counting type radiation image detector is capable of counting the number of photons of radiation incident on the radiation image detector for each of a plurality of energy bands. Such a photo-counting type radiation image detector is already known, such as that described in JP 2011-24773 A, for example.

Further, the substrate functions as a support of the photoelectric conversion panel, and can be the same as the support used in the scintillator panel according to an embodiment of the present invention described above.

As an example of the photoelectric conversion element, a planar light receiving element or the like can also be adopted as described in JP 2015-230175 A. For example, the substrate may have a configuration in which a plurality of light receiving elements are two-dimensionally arranged on an insulating substrate. Specifically; the photoelectric conversion panel is embedded in AeroDR (manufactured by KONICA MINOLTA JAPAN, INC.), PaxScan (FPD: 2520 manufactured by Varian Medical Systems, Inc.), and the like.

The insulating substrate can also serve as a support for the scintillator member, and the element itself may be curved so as to follow the inclined structure and curvature of the scintillator member. In such a case, a glass plate or a polymer material is preferable. From the viewpoint of easiness of bending, a polymer material, particularly a resin film is preferable, and a polyimide film is particularly preferable from the viewpoint of heat resistance.

Further, the photoelectric conversion panel may further include various components that can be possessed by the photoelectric conversion panel constituting a known radiation detector such as a memory unit that stores intensity information of radiation such as an X-ray converted into an electric signal and an image signal based on position information, a power supply unit that supplies electric power necessary for driving the photoelectric conversion panel, and a communication output unit that extracts image information to the outside.

In the photoelectric conversion panel, the photoelectric conversion element is arranged on a substrate including a material such as amorphous silicon so as to have a predetermined pitch. There may be a case where a plurality of photoelectric conversion panels are joined as means for increasing the area of the photoelectric conversion panel. The arrangement of such a photoelectric conversion panel is referred to as tiling.

The joint of the photoelectric conversion panel by tiling is a non-light receiver.

When the width of the sectioned scintillator is reduced to be smaller than the width of the non-light receiving portion of the photoelectric conversion panel, as shown in FIG. 4, a portion which cannot receive light is generated directly under the non-scintillator layer, and since the light is not directly received by the non-light receiver, the information of that portion is not reflected in the image.

Therefore, as shown in FIG. 1, in an embodiment of the present invention, a layer made of a light transmissive material is disposed between the sectioned scintillator and the photoelectric conversion panel, and the light is diffused and easily transmitted to the photoelectric conversion element.

Light Transmissive Material Layer

In an embodiment of the present invention, a light transmissive material layer is provided between the sectioned scintillator and the photoelectric conversion panel. Due to this material layer, light emitted from the scintillator is diffused and light can be received by the photoelectric conversion element.

Usually, the light transmissive material layer includes an organic resin. The light transmissive material layer may have a multilayer structure or may include an air layer, an adhesive functional layer, and the like.

The light transmissive material layer is formed so as to be in close contact with the surface of the sectioned scintillator and the surface of the photoelectric conversion panel.

The thickness of the light transmissive material layer is preferably 10% or more with respect to the width of the non-light receiver present in the photoelectric conversion panel in order to favorably diffuse the light emitted from the scintillator, but is more preferably 30% or more, and further preferably 100% or more.

The component constituting the light transmissive material layer is not particularly limited as long as the object of an embodiment of the present invention is not impaired, but a thermosetting resin, a hot-melt sheet or a pressure-sensitive adhesive sheet is preferred.

As the thermosetting resin, liar example, a resin formed of acrylic, epoxy, silicone or the like, as a main component, can be cited. Above all, resins formed of acrylic resin and silicone-based resin as a main component is preferable from the viewpoint of low temperature thermal curing. Examples of commercially available products include methyl silicone-based JCR 6122 manufactured by Dow Corning Toray Co., Ltd., and the like.

The hot-melt sheet in an embodiment of the present invention is a sheet-shaped adhesive resin (hereinafter referred to as a hot melt resin) which is solid at room temperature and is made of a nonvolatile thermoplastic material without containing water or a solvent. By inserting the hot melt sheet between adherends and melting the hot melt sheet at a temperature equal to or higher than the melting point and solidifying at a temperature equal to or lower than the melting point, the adherends can be joined to each other via the hot melt sheet. Since the hot-melt resin does not contain a polar solvent, a solvent, and water, a phosphor layer does not deliquesce even when the hot-melt resin comes in contact with the deliquescent phosphor layer (for example, a phosphor layer having a columnar crystal structure including an alkali halide), so that the hot melt resin is suitable for joining the photoelectric conversion element and the phosphor layer. Furthermore, since the hot melt sheet does not contain residual volatiles, shrinkage due to drying is small, and a gap filling property and dimensional stability are excellent.

Specific examples of the hot-melt sheet include sheets formed mainly of resins such as polyolefin-based resin, polyamide-based resin, polyester-based resin, polyurethane-based resin, acrylic resin, or EVA-based resin, depending on a main component. Among them, the hot melt sheet formed mainly of resins such as polyolefin-based resin, EVA-based resin, acrylic resin are preferable from the viewpoint of light permeability and adhesiveness.

The light transmissive material layer may be the pressure-sensitive adhesive sheet. Specific examples of the pressure-sensitive adhesive sheet include sheets formed of resins such as acrylic resin, urethane-based resin, rubber-based resin, silicone-based resin and the like as a main component. Among them, from the viewpoints of light transmittance and adhesiveness, a pressure sensitive adhesive sheet formed of resins such as acrylic resin and silicone-based resin is preferable.

In the case of thermosetting resin, the light transmissive material layer is applied onto the scintillator layer or the photoelectric conversion element by a technique such as spin coating, screen printing, dispenser, or the like.

In the case of the hot melt sheet, the light transmissive material layer is formed by inserting the hot melt sheet between the scintillator layer and the photoelectric conversion element and heating under reduced pressure.

The pressure-sensitive adhesive sheet is laminated by a lamination device or the like.

Furthermore, the light transmissive material layer may include a fiber optic plate (FOP). The FOP is an optical device with a bundle of several μm optical fibers, and can propagate the incident tight to the photoelectric conversion element with high efficiency and low distortion. Furthermore, the FOP has a high radiation shielding effect and can prevent radiation damage to various elements constituting the photodetector used for the radiographic image converter.

For the FOP, it is possible to select a commercially available FOP from its radiation shielding rate, visible light transmittance and the like. The FOP is joined to the sectioned scintillator and the photoelectric conversion panel via a connecting member. As the connecting member, a double-sided pressure-sensitive adhesive sheet, a liquid curing type adhesive material, an adhesive, or the like is used. Particularly preferably, an optical pressure-sensitive adhesive sheet or an adhesive material is used. As the adhesive material, either an organic material or an inorganic material may be used. For example, adhesive materials such as acrylic adhesive material, epoxy-based adhesive material, silicone-based adhesive material, natural rubber-based adhesive material, silica-based adhesive material, urethane-based adhesive material, ethylene-based adhesive material, polyolefin-based adhesive material, polyester-based adhesive material, polyurethane-based adhesive material, polyamide-based adhesive material, cellulose-based adhesive material and the like are appropriately used. These can be used either singly or in combination. In addition, as the structure of the pressure-sensitive adhesive sheet, a sheet in which an adhesive layer is formed on both sides of a core material such as PET, a sheet formed as a single-layer pressure-sensitive adhesive layer without a core material, and the like are used.

The light transmissive material layer is transparent so that light emitted from the scintillator layer by application of the radiation reaches the photoelectric conversion element, and it is preferable that the transmittance of light is high transmittance of 90% or more.

According to an embodiment of the present invention, even if there is the non-light receiver in the photoelectric conversion panel, since the light transmissive resin layer exists between the light transmissive material layer and the scintillator layer, the light emission is diffused and can, be received. For this reason, light emission from the scintillator is easily transmitted to the sensor, and image quality is improved. Furthermore, according to an embodiment of the present invention, it is also possible to achieve large area and thick film formation, which has been conventionally difficult, and it is possible to arbitrarily adjust the laminated pitch.

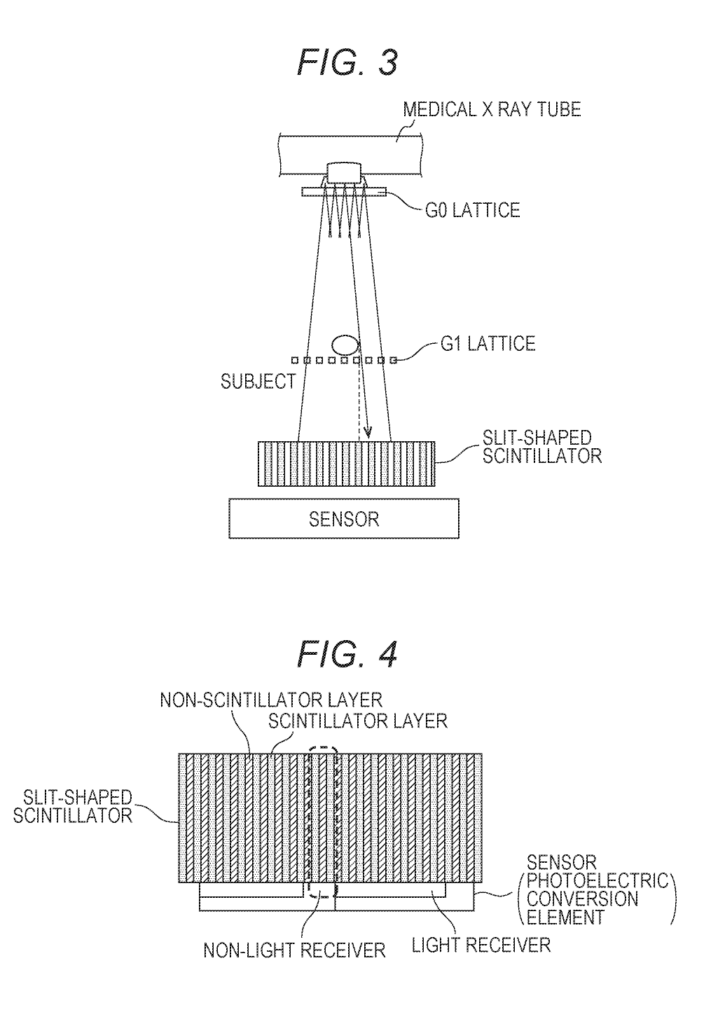

Therefore, the radiation conversion panel according to an embodiment of the present invention can be suitably used for a Talbot imaging device. For example, as in the Talbot imaging device shown in FIG. 3, the radiation conversion panel can be suitably employed as a scintillator having a sectioned structure and a photoelectric conversion panel by removing the G2 lattice. Incidentally, the Talbot imaging device is described in detail in JP 2016-220865 A, JP 2016-220787 A, JP 2016-209017 A, JP 2016-150173 A, and the like.

According to an embodiment of the present invention, with the scintillator panel having the sectioned structure, by providing a layer made of a light transmissive material between the scintillator panel and the photoelectric conversion panel, a gap is generated between the scintillator panel and the photoelectric conversion panel. Even when the non-light receiver is present in the photoelectric conversion panel, the light emission of the scintillator diffuses and is easily transmitted to the sensor, thereby improving the image quality. Such a radiation conversion panel can be used for a Talbot imaging device.

The radiation conversion panel of the present invention has high brightness and is suitable for large area and thick film formation. Therefore, it becomes possible to take high-pressure photography, and it is also possible to photograph a thick subject such as a thoracoabdominal part, thigh part, elbow joint, knee joint, and hip joint.

Conventionally, in diagnostic imaging of cartilage, MRI is mainstream, and there are disadvantages of high photographing cost and long photographing time because of using large equipment. On the other hand, according to an embodiment of the present invention, it is possible to photograph soft tissue such as cartilage, muscle tendon, ligament and visceral tissue with a faster x-ray image at lower cost. Therefore, it can be widely applied to orthopedic diseases such as rheumatoid arthritis and osteoarthritis of knee and image diagnosis of soft tissues such as breast cancer.

Although embodiments of the present invention have been described and illustrated in detail, the disclosed embodiments are made for purposes of illustration and example only and not limitation. The scope of the present invention should be interpreted by terms of the appended claims.

What is claimed is:

1. A radiation conversion panel comprising:
a scintillator panel having a sectioned structure; and
a photoelectric conversion panel, the scintillator panel and the photoelectric conversion panel being disposed so as to be opposed to each other, the scintillator having a width smaller than a width of a non-light receiver present in the photoelectric conversion panel, wherein
a layer made of a light transmissive material is disposed between the scintillator panel and the photoelectric conversion panel.

2. The radiation conversion panel according to claim 1, wherein the photoelectric conversion panel is formed by joining a plurality of photoelectric conversion panels.

3. The radiation conversion panel according to claim 1, wherein a sectioned structure of the scintillator is a slit shape.

4. A Talbot imaging device using the radiation conversion panel according to claim 1.
